SEQUENCE LISTING (23 sequences)

SEQ ID NO: 1 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 171 aa, Homo sapiens
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVR

SEQ ID NO: 8 - protein, 150 aa, Homo sapiens
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSR

SEQ ID NO: 9 - protein, 121 aa, Homo sapiens
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPK

SEQ ID NO: 10 - protein, 67 aa, Homo sapiens
AEPRQEFEVMEDHAGTYGLGDRKDQGGYTMHQDQEGDTDAGLKAEEAGIGDTPSLEDEAAGHVTQAR

SEQ ID NO: 11 - protein, 10 aa, Artificial Sequence
  source: /note=“Description of Artificial Sequence      Synthetic peptide”
AGTYGLGDRK

SEQ ID NO: 12 - protein, 117 aa, Artificial Sequence
  source: /note=“Description of Artificial Sequence      Synthetic polypeptide”
EVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSS

SEQ ID NO: 13 - protein, 112 aa, Artificial Sequence
  source: /note=“Description of Artificial Sequence      Synthetic polypeptide”
DVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIK

SEQ ID NO: 14 - protein, 444 aa, Artificial Sequence
  source: /note=“Description of Artificial Sequence      Synthetic polypeptide”
EVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 219 aa, Artificial Sequence
  source: /note=“Description of Artificial Sequence      Synthetic polypeptide”
DVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, Artificial Sequence
  source: /note=“Description of Artificial Sequence      Synthetic peptide”
KYGMS

SEQ ID NO: 17 - protein, 17 aa, Artificial Sequence
  source: /note=“Description of Artificial Sequence      Synthetic peptide”
TISSSGSRTYYPDSVKG

SEQ ID NO: 18 - protein, 8 aa, Artificial Sequence
  source: /note=“Description of Artificial Sequence      Synthetic peptide”
SWDGAMDY

SEQ ID NO: 19 - protein, 16 aa, Artificial Sequence
  source: /note=“Description of Artificial Sequence      Synthetic peptide”
KSSQSIVHSNGNTYLE

SEQ ID NO: 20 - protein, 7 aa, Artificial Sequence
  source: /note=“Description of Artificial Sequence      Synthetic peptide”
KVSNRFS

SEQ ID NO: 21 - protein, 9 aa, Artificial Sequence
  source: /note=“Description of Artificial Sequence      Synthetic peptide”
FQGSLVPWA

SEQ ID NO: 22 - protein, 22 aa, Artificial Sequence
  source: /note=“Description of Artificial Sequence      Synthetic peptide”
MDMRVPAQLLGLLLLWFPGSRC

SEQ ID NO: 23 - protein, 22 aa, Artificial Sequence
  source: /note=“Description of Artificial Sequence      Synthetic peptide”
MDMRVPAQLLGLLLLWLPGARC

(19) United States
(12) Patent Application Publication
(10) Pub. No.: US 2019/0135905 A1
(43) Pub. Date: May 9, 2019

(54) COMPOSITIONS AND METHODS FOR TREATING TAUOPATHIES

(71) Applicant: Bristol-Myers Squibb Company, Princeton, NJ (US)

(72) Inventors: Giridhar S. Tirucherai, Kendall Park, NJ (US); Michael Frans Burgess, Chester, NJ (US); Masano Tlaneci Huang, Cranbury, NJ (US); Lori S. Burton, Robbinsville, NJ (US); Yong Quan, Dublin, CA (US)

(73) Assignee: Bristol-Myers Squibb Company, Princeton, NJ (US)

(21) Appl. No.: 16/007,301

(22) Filed: Jun. 13, 2018

Related U.S. Application Data
(60) Provisional application No. 62/521,155, filed on Jun. 16, 2017.

(51) Int. Cl. C07K 16/18; A61P 25/28; A61K 9/00

(52) U.S. Cl. CPC C07K 16/18; A61K 38/00; A61K 9/0019; A61P 25/28

(57) ABSTRACT

Dosage regimens and formulations of anti-human tau antibodies are provided. These formulations and dosage regimens find use in the treatment of tauopathies such as progressive supranuclear palsy and Alzheimer's disease.

### CROSS-REFERENCE TO RELATED APPLICATION(S)

This application claims the benefit of U.S. Application No. 62/521,155, filed on Jun. 16, 2017. The disclosure of the prior application is incorporated herein by reference.

### FIELD

The present application relates generally to dosage regimens and formulations for the clinical use of anti-tau antibodies.

### BACKGROUND

Protein accumulation, modifications, and aggregation are pathological aspects of numerous neurodegenerative diseases. Pathologically modified and aggregated tau including hyperphosphorylated tau conformers are an invariant hallmark of tauopathies and correlate with disease severity.

The microtubule associated protein tau is abundant in the central nervous system and is produced primarily by neurons. Tau promotes the assembly of, maintains the structure of, and stabilizes microtubules. The tau proteins are derived from alternative mRNA splice variants that originate from a single gene and result in mature proteins that vary in size from 352 to 441 amino acids. While the fetal brain contains a single tau isoform (Tau-352), six tau isoforms exist in the adult human brain. They differ from one another in having three or four microtubule binding repeats of 31-32 amino acids each, and two, one, or no amino terminal inserts of 29 amino acids each.

Tauopathies are a class of neurodegenerative diseases resulting from the pathological aggregation of Tau protein in so-called neurofibrillary tangles (NFT) in the brain. Some examples of tauopathies include progressive supranuclear palsy, Alzheimer's disease, frontotemporal dementia (FTD), corticobasal degeneration, and frontotemporal lobar degeneration.

There is a need in the art for methods of treating tauopathies. In order to treat the growing numbers of patients with tauopathies, there is a need for a therapeutic antibody against tau and appropriate dosage regimens and formulations for the clinical use of such an anti-human tau antibody.

### SUMMARY

This disclosure relates, in part, to dosage regimens and formulations of anti-human tau antibodies or tau-binding fragments thereof and their use in the treatment of a tauopathy.

In one aspect, this disclosure provides a method of treating a tauopathy in a human subject in need thereof. The method involves administering to the human subject an anti-human tau antibody at a fixed dose of 2000 mg. The anti-human tau antibody comprises an immunoglobulin heavy chain variable region (VH) and an immunoglobulin light chain variable region (VL). The VH comprises VH complementarity determining regions (VH-CDRs), wherein VH-CDR1 comprises or consists of the amino acid sequence of SEQ ID NO:16; VH-CDR2 comprises or consists of the amino acid sequence of SEQ ID NO:17; and VH-CDR3 comprises or consists of the amino acid sequence of SEQ ID NO:18. The VL comprises VL-CDRs, wherein VL-CDR1 comprises or consists of the amino acid sequence of SEQ ID NO:19; VL-CDR2 comprises or consists of the amino acid sequence of SEQ ID NO:20; and VL-CDR3 comprises or consists of the amino acid sequence of SEQ ID NO:21. In certain instances the anti-human tau antibody is administered to the human subject intravenously. In certain cases, the fixed dose of 2000 mg of the anti-human tau antibody is administered once every four weeks.

In another aspect, provided herein is a method of treating a tauopathy in a human subject in need thereof. The method involves administering to the human subject an anti-human tau antibody at a fixed dose of 150 mg. The anti-human tau antibody comprises an immunoglobulin heavy chain variable region (VH) and an immunoglobulin light chain variable region (VL). The VH comprises VH complementarity determining regions (VH-CDRs), wherein VH-CDR1 comprises or consists of the amino acid sequence of SEQ ID NO:16; VH-CDR2 comprises or consists of the amino acid sequence of SEQ ID NO:17; and VH-CDR3 comprises or consists of the amino acid sequence of SEQ ID NO:18. The VL comprises VL-CDRs, wherein VL-CDR1 comprises or consists of the amino acid sequence of SEQ ID NO:19; VL-CDR2 comprises or consists of the amino acid sequence of SEQ ID NO:20; and VL-CDR3 comprises or consists of the amino acid sequence of SEQ ID NO:21. In certain instances the anti-human tau antibody is administered to the human subject intravenously. In certain cases, the fixed dose of 150 mg of the anti-human tau antibody is administered once every four weeks.

In another aspect, provided herein is a method of treating a tauopathy in a human subject in need thereof. The method involves administering to the human subject an anti-human tau antibody at a fixed dose of 210 mg. The anti-human tau antibody comprises an immunoglobulin heavy chain variable region (VH) and an immunoglobulin light chain variable region (VL). The VH comprises VH complementarity determining regions (VH-CDRs), wherein VH-CDR1 comprises or consists of the amino acid sequence of SEQ ID NO:16; VH-CDR2 comprises or consists of the amino acid sequence of SEQ ID NO:17; and VH-CDR3 comprises or consists of the amino acid sequence of SEQ ID NO:18. The VL comprises VL-CDRs, wherein VL-CDR1 comprises or consists of the amino acid sequence of SEQ ID NO:19; VL-CDR2 comprises or consists of the amino acid sequence of SEQ ID NO:20; and VL-CDR3 comprises or consists of the amino acid sequence of SEQ ID NO:21. In certain instances the anti-human tau antibody is administered to the human subject intravenously. In certain cases, the fixed dose of 210 mg of the anti-human tau antibody is administered once every four weeks.

In another aspect, provided herein is a method of treating a tauopathy in a human subject in need thereof. The method involves administering to the human subject an anti-human tau antibody at a fixed dose of 2100 mg. The anti-human tau antibody comprises an immunoglobulin heavy chain variable region (VH) and an immunoglobulin light chain variable region (VL). The VH comprises VH complementarity determining regions (VH-CDRs), wherein VH-CDR1 comprises or consists of the amino acid sequence of SEQ ID NO:16; VH-CDR2 comprises or consists of the amino acid sequence of SEQ ID NO:17; and VH-CDR3 comprises or consists of the amino acid sequence of SEQ ID NO:18. The VL comprises VL-CDRs, wherein VL-CDR1 comprises or consists of the amino acid sequence of SEQ ID NO:19; VL-CDR2 comprises or consists of the amino acid sequence of SEQ ID NO:20; and VL-CDR3 comprises or consists of the amino acid sequence of SEQ ID NO:21. In certain instances the anti-human tau antibody is administered to the human subject intravenously. In certain cases, the fixed dose of 2100 mg of the anti-human tau antibody is administered once every four weeks.

In one aspect, provided herein is a method of treating a tauopathy in a human subject in need thereof. The method involves administering to the human subject an anti-human tau antibody at a fixed dose of 4200 mg. The anti-human tau antibody comprises an immunoglobulin heavy chain variable region (VH) and an immunoglobulin light chain variable region (VL). The VH comprises VH complementarity determining regions (VH-CDRs), wherein VH-CDR1 comprises or consists of the amino acid sequence of SEQ ID NO:16; VH-CDR2 comprises or consists of the amino acid sequence of SEQ ID NO:17; and VH-CDR3 comprises or consists of the amino acid sequence of SEQ ID NO:18. The VL comprises VL-CDRs, wherein VL-CDR1 comprises or consists of the amino acid sequence of SEQ ID NO:19; VL-CDR2 comprises or consists of the amino acid sequence of SEQ ID NO:20; and VL-CDR3 comprises or consists of the amino acid sequence of SEQ ID NO:21. In certain instances the anti-human tau antibody is administered to the human subject intravenously. In certain cases, the fixed dose of 4200 mg of the anti-human tau antibody is administered once every four weeks.

The following embodiments apply to all of the above aspects. In some instances, the tauopathy is progressive supranuclear palsy, Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, globular glial tauopathy, subacute sclerosing panencephalitis, Tangle only dementia, multi-infarct dementia, stroke, chronic traumatic encephalopathy, traumatic brain injury, concussion, seizures, epilepsy, or acute lead encephalopathy. In one instance, the tauopathy is progressive supranuclear palsy. In another instance, the tauopathy is Alzheimer's disease. In certain cases, the VH of the anti-human tau antibody comprises or consists of SEQ ID NO:12, and the VL of the anti-human tau antibody comprises or consists of SEQ ID NO:13. In certain instances, the anti-human tau antibody comprises a heavy chain and a light chain, wherein the heavy chain comprises or consists of SEQ ID NO:14, and the light chain comprises or consists of SEQ ID NO:15.

In another aspect, this disclosure features a pharmaceutical composition comprising an anti-human tau antibody. The pharmaceutical composition comprises an anti-human tau antibody at a concentration of 50 mg/ml or 60 mg/ml; histidine at a concentration of 20 mM, sucrose at a concentration of 250 mM, polysorbate-80 at a concentration of 0.05% (w/v). In some instances, the pharmaceutical composition further comprises 50 μM diethylenetriamine pentaacetic acid (DTPA). The anti-human tau antibody comprises a VH and a VL. The VH comprises a VH-CDR1 comprising or consisting of the amino acid sequence of SEQ ID NO:16; a VH-CDR2 comprising or consisting of the amino acid sequence of SEQ ID NO:17; and a VH-CDR3 comprising or consisting of the amino acid sequence of SEQ ID NO:18. The VL comprises a VL-CDR1 comprising or consisting of the amino acid sequence of SEQ ID NO:19; a VL-CDR2 comprising or consisting of the amino acid sequence of SEQ ID NO:20; and a VL-CDR3 comprising or consisting of the amino acid sequence of SEQ ID NO:21. The pharmaceutical composition has a pH of 6.0.

In some embodiments, the VH of the anti-human tau antibody comprises or consists of SEQ ID NO:12 and the VL of the anti-human tau antibody comprises or consists of SEQ ID NO:13. In some embodiments, the anti-human tau antibody comprises a heavy chain and a light chain, wherein the heavy chain comprises or consists of SEQ ID NO:14 and the light chain comprises or consists of SEQ ID NO:15. In some embodiments, the pharmaceutical composition is used in for treating a tauopathy in a human subject in need thereof by intravenously administering to the human subject any of the above-described pharmaceutical compositions. In some embodiments, the anti-human tau antibody of the pharmaceutical composition is administered to a human subject at a fixed dose of 150 mg once every four weeks. In other embodiments, the anti-human tau antibody of the pharmaceutical composition is administered to a human subject at a fixed dose of 210 mg once every four weeks. In yet other embodiments, the anti-human tau antibody of the pharmaceutical composition is administered to a human subject at a fixed dose of 700 mg once every four weeks. In further embodiments, the anti-human tau antibody of the pharmaceutical composition is administered to a human subject at a fixed dose of 2000 mg once every four weeks. In other embodiments, the anti-human tau antibody of the pharmaceutical composition is administered to a human subject at a fixed dose of 2100 mg once every four weeks. In another embodiment, the anti-human tau antibody of the pharmaceutical composition is administered to a human subject at a fixed dose of 4200 mg once every four weeks. In certain instances, the pharmaceutical composition is administered for at least 12 weeks (e.g., 12 weeks, 16 weeks, 20 weeks, 24 weeks, 30 weeks, 32 weeks, 36 weeks, 40 weeks, 48 weeks, 52 weeks). In certain instances, the tauopathy is progressive supranuclear palsy, Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, globular glial tauopathy, subacute sclerosing panencephalitis, Tangle only dementia, multi-infarct dementia, stroke, chronic traumatic encephalopathy, traumatic brain injury, concussion, seizures, epilepsy, or acute lead encephalopathy. In one embodiment, the tauopathy is progressive supranuclear palsy. In another embodiment, the tauopathy is Alzheimer's disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF DRAWINGS

FIG. 1 provides the sequences of different forms of extracellular Tau (eTau), eTau1 (SEQ ID NO:7); eTau2 (SEQ ID NO:8), eTau3 (SEQ ID NO:9), and eTau4 (SEQ ID NO:10), compared to the human Tau-441 isoform (2N4R) sequence (SEQ ID NO:6).

FIG. 2 is a schematic representation of the study design for the single ascending dose study described in Example 1.

FIG. 3 is a graph depicting the exposure-response model (Bayesian Eₘₐₓ) of CSF concentration versus eTau suppression.

FIG. 4 is a schematic representation of the study design for the multiple ascending dose study described in Example 3.

FIG. 5 is a table providing the baseline demographic characteristics of the patients in the multiple ascending dose study described in Example 3.

FIG. 6 is a table providing a summary of the adverse events for the multiple ascending dose study described in Example 3.

FIG. 7 is a table providing a summary of the serum PK parameters for BIIB092 (at day 57 of the study).

FIG. 8 is a graphical depiction of the mean change in eTau concentrations over time. There was a dose-dependent relationship between BIIB092 dose and the extent of eTau suppression in the CSF.

FIG. 9 is a table providing CSF free eTau as a percentage of baseline with BIIB092 dose.

### DETAILED DESCRIPTION

This disclosure features dosage regimens and formulations of anti-human tau antibodies and tau-binding fragments thereof and their use in the treatment of tauopathies (e.g., disorders related to aggregates of tau such as progressive supranuclear palsy, Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, subacute sclerosing panencephalitis, Tangle only dementia, multi-infarct dementia, stroke, chronic traumatic encephalopathy, traumatic brain injury, concussion, seizures, epilepsy, and acute lead encephalopathy).

### Tau

Tau is a protein that plays a critical role in the pathogenesis of several disorders collectively referred to as tauopathies. There are several different isoforms of the microtubule-associated protein, which are provided below:

Isoform Fetal-tau of 352aa
(SEQ ID NO: 1)
MAEPRQEFEVMEDHAGTYGLGDRKDQGGYTMHQDQEGDTDAGLKAEEAGIGDTPSLEDE
AAGHVTQARMVSKSKDGTGSDDKKAKGADGKTKIATPRGAAPPGQKGQANATRIPAKTP
PAPKTPPSSGEPPKSGDRSGYSSPGSPGTPGSRSRTPSLPTPPTREPKKVAVVRTPPKS
PSSAKSRLQTAPVPMPDLKNVKSKIGSTENLKHQPGGGKVQIVYKPVDLSKVTSKCGSL
GNIHHKPGGGQVEVKSEKLDFKDRVQSKIGSLDNITHVPGGGNKKIETHKLTFRENAKA
KTDHGAEIVYKSPVVSGDTSPRHLSNVSSTGSIDMVDSPQLATLADEVSASLAKQGL
Isoform Tau-B of 381aa
(SEQ ID NO: 2)
MAEPRQEFEVMEDHAGTYGLGDRKDQGGYTMHQDQEGDTDAGLKESPLQTPTEDGSEEP
GSETSDAKSTPTAEAEEAGIGDTPSLEDEAAGHVTQARMVSKSKDGTGSDDKKAKGADG
KTKIATPRGAAPPGQKGQANATRIPAKTPPAPKTPPSSGEPPKSGDRSGYSSPGSPGTP
GSRSRTPSLPTPPTREPKKVAVVRTPPKSPSSAKSRLQTAPVPMPDLKNVKSKIGSTEN
LKHQPGGGKVQIVYKPVDLSKVTSKCGSLGNIHHKPGGGQVEVKSEKLDFKDRVQSKIG
SLDNITHVPGGGNKKIETHKLTFRENAKAKTDHGAEIVYKSPVVSGDTSPRHLSNVSST
GSIDMVDSPQLATLADEVSASLAKQGL
Isoform Tau-C of 410aa
(SEQ ID NO: 3)
MAEPRQEFEVMEDHAGTYGLGDRKDQGGYTMHQDQEGDTDAGLKESPLQTPTEDGSEEP
GSETSDAKSTPTAEDVTAPLVDEGAPGKQAAAQPHTEIPEGTTAEEAGIGDTPSLEDEA
AGHVTQARMVSKSKDGTGSDDKKAKGADGKTKIATPRGAAPPGQKGQANATRIPAKTPP
APKTPPSSGEPPKSGDRSGYSSPGSPGTPGSRSRTPSLPTPPTREPKKVAVVRTPPKSP
SSAKSRLQTAPVPMPDLKNVKSKIGSTENLKHQPGGGKVQIVYKPVDLSKVTSKCGSLG
NIHHKPGGGQVEVKSEKLDFKDRVQSKIGSLDNITHVPGGGNKKIETHKLTFRENAKAK
TDHGAEIVYKSPVVSGDTSPRHLSNVSSTGSIDMVDSPQLATLADEVSASLAKQGL
Isoform Tau-D of 383aa
(SEQ ID NO: 4)
MAEPRQEFEVMEDHAGTYGLGDRKDQGGYTMHQDQEGDTDAGLKAEEAGIGDTPSLEDE
AAGHVTQABMVSKSKDGTGSDDKKAKGADGKTKIATPRGAAPPGQKGQANATRIPAKTP
PAPKTPPSSGEPPKSGDRSGYSSPGSPGTPGSRSRTPSLPTPPTREPKKVAVVRTPPKS
PSSAKSRLQTAPVPMPDLKNVKSKIGSTENLKHQPGGGKVQIINKKLDLSNVQSKCGSK
DNIKHVPGGGSVQIVYKPVDLSKVTSKCGSLGNIHHKPGGGQVEVKSEKLDFKDRVQSK
IGSLDNITHVPGGGNKKIETHKLTFRENAKAKTDHGAEIVYKSPVVSGDTSPRHLSNVS
STGSIDMVDSPQLATLADEVSASLAKQGL
Isoform Tau-E of 412aa
(SEQ ID NO: 5)
MAEPRQEFEVMEDHAGTYGLGDRKDQGGYTMHQDQEGDTDAGLKESPLQTPTEDGSEEP
GSETSDAKSTPTAEAEEAGIGDTPSLEDEAAGHVTQARMVSKSKDGTGSDDKKAKGADG
KTKIATPRGAAPPGQKGQANATRIPAKTPPAPKTPPSSGEPPKSGDRSGYSSPGSPGTP
GSRSRTPSLPTPPTREPKKVAVVRTPPKSPSSAKSRLQTAPVPMPDLKNVKSKIGSTEN
LKHQPGGGKVQIINKKLDLSNVQSKCGSKDNIKHVPGGGSVQIVYKPVDLSKVTSKCGS
LGNIHHKPGGGQVEVKSEKLDFKDRVQSKIGSLDNITHVPGGGNKKIETHKLTFRENAK
AKTDHGAEIVYKSPVVSGDTSPRHLSNVSSTGSIDMVDSPQLATLADEVSASLAKQGL
Isoform Tau-F (2N4R) of 441aa
(SEQ ID NO: 6)
MAEPRQEFEVMEDHAGTYGLGDRKDQGGYTMHQDQEGDTDAGLKESPLQTPTEDGSEEP
GSETSDAKSTPTAEDVTAPLVDEGAPGKQAAAQPHTEIPEGTTAEEAGIGDTPSLEDEA
AGHVTQARMVSKSKDGTGSDDKKAKGADGKTKIATPRGAAPPGQKGQANATRIPAKTPP
APKTPPSSGEPPKSGDRSGYSSPGSPGTPGSRSRTPSLPTPPTREPKKVAVVRTPPKSP
SSAKSRLQTAPVPMPDLKNVKSKIGSTENLKHQPGGGKVQIINKKLDLSNVQSKCGSKD
NIKHVPGGGSVQIVYKPVDLSKVTSKCGSLGNIHHKPGGGQVEVKSEKLDFKDRVQSKI
GSLDNITHVPGGGNKKIETHKLTFRENAKAKTDHGAEIVYKSPVVSGDTSPRHLSNVSS
TGSIDMVDSPQLATLADEVSASLAKQGL

Intracellular tau levels are increased in the brains of Alzheimer's disease patients when compared to non-demented controls (Barton, Am J. Pathol., 137:497-502 (1990); Khatoon, J. Neurochem., 59:750-753 (1992)). This increase in the levels of intracellular tau is believed to be toxic to neurons since a reduction in the amount of intracellular tau has been shown to be protective in mouse models of neurodegeneration (Rapoport et al., Proc. Natl. Acad. Sci. USA, 99:6364-6369 (2002); Robertson et al., Science, 316:750-754 (2007)), and thus reducing the amount of intracellular tau can be therapeutically beneficial.

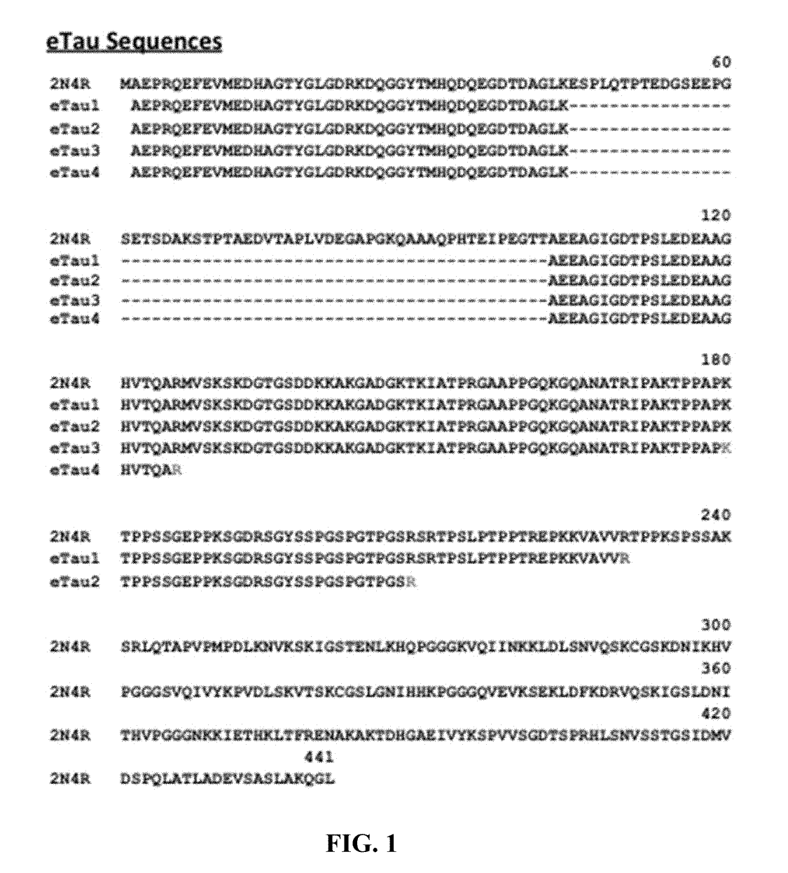

Secreted N-terminally truncated tau species, designated extracellular Tau (eTau), have been identified. “eTau” as used herein, encompasses any Tau polypeptide that can be detected in cerebrospinal fluid (CSF) or interstitial fluid (ISF). In some embodiments, eTau is a polypeptide having a sequence set forth in one of SEQ ID NOS.: 7-10 of FIG. 1. The eTau species varies from 171 amino acids for eTau1 to 67 amino acids for eTau4. Although tau lacks a signal sequence, tau has been found released into culture medium from neuroblastoma cells, tau-expressing non-neuronal cells, induced pluripotent stem cell-derived human neurons, and mouse primary neurons. Thus, tau may be secreted unconventionally or associated with exosomes or other membrane vesicles. eTau has also been detected in the brain ISF of both wild type and P301S tau-expressing mice in microdialysis studies. It has also been seen in the brain ISF of patients following traumatic brain injury. eTau has been shown to regulate Aβ production and to increase neuronal hyperactivity (Bright et al., Neurobiology and Aging, 36:693-709 (2015)). Treatment with an eTau-neutralizing antibody reduces eTau-mediated neuronal hyperactivity. See, e.g., WO 2014/02877. It has been proposed that the eTau-driven neuronal hyperactivity increase leads not only to increased Aβ secretion but also to a further increase in eTau secretion and thus, eTau and Aβ create a feed forward disease mechanism that perpetuates the disease. Thus, neutralizing eTau can inhibit the spread of tau and tau pathology in the brain, reduce central nervous system Aβ levels and the resulting neuronal hyperactivity, and potentially slow the clinical progression into dementia.

### Anti-Human Tau Antibodies

One way of neutralizing tau is by using antibodies that bind tau. In certain embodiments, these antibodies bind to an epitope within amino acids 1-25, 1-18, 9-18, 13-24, 15-44, or 15-24 of SEQ ID NO:6. In a specific embodiment, an anti-tau antibody binds to an epitope within AGTYGLGDRK (SEQ ID NO:11).

An exemplary anti-human tau antibody is designated “BIIB092.” BIIB092 is a humanized IgG4/kappa antibody that recognizes human tau. The heavy chain variable domain of the BIIB092 antibody has the following amino acid sequence:

(SEQ ID NO: 12)
EVHLVESGGA LVKPGGSLRL SCAASGFSFS KYGMSWVRQA
PGKGLEWVAT ISSSGSRTYY PDSVKGRFTI SRDNAKNTLY
LQMNSLRAED TAMYYCSISW DGAMDYWGQG TTVTVSS

The light chain variable domain of the BIIB092 antibody has the following amino acid sequence:

(SEQ ID NO: 13)
DVVMTQSPLS LPVTLGQPAS ISCKSSQSIV HSNGNTYLEW
YLQKPGQSPQ LLVYKVSNRF SGVPDRFSGS GSGTDFTLKI
SRVEAEDVGT YYCFQGSLVP WAFGGGTKVE IK

The heavy chain of the BIIB092 antibody has the following amino acid sequence:

(SEQ ID NO: 14)
EVHLVESGGA LVKPGGSLRL SCAASGFSFS KYGMSWVRQA
PGKGLEWVAT ISSSGSRTYY PDSVKGRFTI SRDNAKNTLY
LQMNSLRAED TAMYYCSISW DGAMDYWGQG TTVTVSSAST
KGPSVFPLAP CSRSTSESTA ALGCLVKDYF PEPVTVSWNS
GALTSGVHTF PAVLQSSGLY SLSSVVTVPS SSLGTKTYTV
NVDHKPSNTK VDKRVESKYG PPCPPCPAPE FLGGPSVFLF
PPKPKDTLMI SRTPEVTCVV VDVSQEDPEV QFNWYVDGVE
VHNAKTKPRE EQFNSTYRVV SVLTVLHQDW LNGKEYKCKV
SNKGLPSSIE KTISKAKGQP REPQVYTLPP SQEEMTKNQV
SLTCLVKGFY PSDIAVEWES NGQPENNYKT TPPVLDSDGS
FFLYSRLTVD KSRWQEGNVF SCSVMHEALH NHYTQKSLSL
SLGK

The light chain of the BIIB092 antibody has the following amino acid sequence:

(SEQ ID NO: 15)
DVVMTQSPLS LPVTLGQPAS ISCKSSQSIV HSNGNTYLEW
YLQKPGQSPQ LLVYKVSNRF SGVPDRFSGS GSGTDFTLKI
SRVEAEDVGT YYCFQGSLVP WAFGGGTKVE IKRTVAAPSV
FIFPPSDEQL KSGTASVVCL LNNFYPREAK VQWKVDNALQ
SGNSQESVTE QDSKDSTYSL SSTLTLSKAD YEKHKVYACE
VTHQGLSSPV TKSFNRGEC

In some embodiments, the anti-human tau antibody or tau-binding fragment thereof comprises the three light chain variable domain CDRs of BIIB092. In some embodiments, the anti-human tau antibody or tau-binding fragment thereof comprises the three heavy chain variable domain CDRs of BIIB092. In still other embodiments, the anti-human tau antibody or tau-binding fragment thereof comprises the three heavy chain variable domain CDRs and the three light chain variable domain CDRs of BIIB092. The CDRs can be based on any CDR definition in the art, e.g., the definitions of Kabat, Chothia, Chothia from Abysis, enhanced Chothia/AbM, or based on the contact definition. CDR sequences of BIIB092 are provided in Table 1 below.

TABLE 1
Sequences of the CDRs of BIIB092

| Domain | Amino Acid Sequence |
|---|---|
| VH CDR1 | KYGMS (SEQ ID NO: 16) |
| VH CDR2 | TISSSGSRTYYPDSVKG (SEQ ID NO: 17) |
| VH CDR3 | SWDGAMDY (SEQ ID NO: 18) |
| VL CDR1 | KSSQSIVHSNGNTYLE (SEQ ID NO: 19) |
| VL CDR2 | KVSNRFS (SEQ ID NO: 20) |
| VL CDR3 | FQGSLVPWA (SEQ ID NO: 21) |

In some embodiments, the anti-human tau antibody or tau-binding fragment thereof comprises a VH CDR1 comprising or consisting of the amino acid sequence set forth in SEQ ID NO:16, a VH CDR2 comprising or consisting of the amino acid sequence set forth in SEQ ID NO:17; and a VH CDR3 comprising or consisting of the amino acid sequence set forth in SEQ ID NO:18; a VL CDR1 comprising or consisting of the amino acid sequence set forth in SEQ ID NO:19, a VL CDR2 comprising or consisting of the amino acid sequence set forth in SEQ ID NO:20; and a VL CDR3 comprising or consisting of the amino acid sequence set forth in SEQ ID NO:21.

In some embodiments, the anti-human tau antibody or tau-binding fragment thereof comprises a VH comprising or consisting of the amino acid sequence set forth in SEQ ID NO:12. In some embodiments, the anti-human tau antibody or tau-binding fragment thereof comprises a VL comprising or consisting of the amino acid sequence set forth in SEQ ID NO:13. In some embodiments, the anti-human tau antibody or tau-binding fragment thereof comprises a VH comprising or consisting of the amino acid sequence set forth in SEQ ID NO:12 and a VL comprising or consisting of the amino acid sequence set forth in SEQ ID NO:13.

In some embodiments, the anti-human tau antibody or tau-binding fragment thereof comprises a heavy chain comprising or consisting of the amino acid sequence set forth in SEQ ID NO:14. In some embodiments, the anti-human tau antibody or tau-binding fragment thereof comprises a light chain comprising or consisting of the amino acid sequence set forth in SEQ ID NO:15. In some embodiments, the anti-human tau antibody or tau-binding fragment thereof comprises a heavy chain comprising or consisting of the amino acid sequence set forth in SEQ ID NO:14 and a light chain comprising or consisting of the amino acid sequence set forth in SEQ ID NO:15.

In certain embodiments, the anti-human tau antibody is an IgG antibody. In specific embodiments, the anti-human tau antibody has heavy chain constant region chosen from, e.g., IgG1, IgG2, IgG3, IgG4, IgM, IgA1, IgA2, IgD, and IgE. In one embodiment, the anti-human tau antibody is of the IgG4 isotype. In another embodiment, the anti-human tau antibody is of the IgG1 isotype. In yet another embodiment, the anti-human tau antibody is of the IgG2 isotype. In another embodiment, the anti-human tau antibody is of the IgG3 isotype. In further embodiments, the anti-human tau antibody has a light chain constant region chosen from, e.g., a human kappa light chain constant region or a human lambda light chain constant region. In a certain embodiment, the anti-human tau antibody is an IgG4/human kappa light chain antibody.

In some embodiments, the anti-human tau antibody is a full-length (whole) antibody or substantially full-length. The protein can include at least one, and preferably two, complete heavy chains, and at least one, and preferably two, complete light chains. In some embodiments, the anti-human tau antibody is a tau-binding fragment. In some instances, the tau-binding antibody fragment is a Fab, a Fab′, an F(ab′)2, a Facb, an Fv, a single chain Fv (scFv), a sc(Fv)2, or a diabody.

The heavy chain and light chain of the anti-human tau antibodies disclosed herein may also include signal sequences. The signal sequences can be selected from those known in the art, for example, MDMRVPAQLLGLLLLWFPGSRC (SEQ ID NO:22) or MDMRVPAQLLGLLLLWLPGARC (SEQ ID NO:23).

Antibodies, such as BIIB092, or tau-binding fragments thereof can be made, for example, by preparing and expressing synthetic genes that encode the recited amino acid sequences or by mutating human germline genes to provide a gene that encodes the recited amino acid sequences. Moreover, this antibody and other anti-human tau antibodies can be produced, e.g., using one or more of the following methods.

### Methods of Producing Anti-Human Tau Antibodies

Anti-human tau antibodies or tau-binding fragments may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide or polynucleotides encoding the antibody is/are constructed, introduced into an expression vector or expression vectors, and then expressed in suitable host cells. To improve expression, the nucleotide sequences of the light and heavy chain genes can be recoded without changing (or minimally changing—e.g., removal of a C-terminal residue of the heavy or light chain) the amino acid sequence. The areas for potential recoding include those associated with translation initiation, codon usage, and possible unintended mRNA splicing. Polynucleotides encoding an anti-human tau antibody comprising the VH and/or VL, HC and/or LC of the tau antibodies described herein would be readily envisioned by the ordinarily skilled artisan.

Standard molecular biology techniques are used to prepare the recombinant expression vector(s), transfect the host cells, select for transformants, culture the host cells, and recover the anti-human tau antibody.

If the anti-human tau antibodies or tau-binding fragments are to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QlAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)) (e.g., early simian virus 40 promoter), MMLV-LTR promoter, EF1α promoter (Mizushima et al., Nucleic Acids Res., 18:5322 (1990)), or CMV promoter (e.g., human cytomegalovirus immediate early promoter). In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, the anti-human tau antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell. In a specific embodiment, the mammalian cell is a CHO-DG441 cell.

In an exemplary system for antibody expression, a recombinant expression vector encoding both the anti-human tau antibody heavy chain and the anti-human tau antibody light chain of an anti-human tau antibody (e.g., BIIB092) is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Anti-Human Tau Antibody Formulations

Any of the anti-human tau antibodies described herein can be formulated as a pharmaceutical composition. The pharmaceutical composition can comprise 10 mg/mL, 60 mg/mL or 50 mg/mL of an anti-human tau antibody described herein. In a particular embodiment, the pharmaceutical composition comprises 50 mg/mL of an anti-human tau antibody described herein. In addition, the pharmaceutical composition includes histidine at a concentration of 20 mM, sucrose at a concentration of 250 mM, and polysorbate-80 at a concentration of 0.05% (w/v). In certain cases, the pharmaceutical composition further includes 50 μM diethylenetriamine pentaacetic acid (DTPA). The pharmaceutical composition has a pH of 6.0.

In some instances, the anti-human tau antibody of the pharmaceutical composition comprises an immunoglobulin heavy chain variable region (VH) comprising VH complementarity determining regions (VH-CDRs) and an immunoglobulin light chain variable region (VL) comprising VL-CDRs. The VH-CDR1 comprises or consists of the amino acid sequence of SEQ ID NO:16; VH-CDR2 comprises or consists of the amino acid sequence of SEQ ID NO:17; and VH-CDR3 comprises or consists of the amino acid sequence of SEQ ID NO:18. The VL-CDR1 comprises or consists of the amino acid sequence of SEQ ID NO:19; VL-CDR2 comprises or consists of the amino acid sequence of SEQ ID NO:20; and VL-CDR3 comprises or consists of the amino acid sequence of SEQ ID NO:21.

In certain cases, the anti-human tau antibody of the pharmaceutical composition comprises a VH comprising or consisting of SEQ ID NO:12. In certain cases, the anti-human tau antibody of the pharmaceutical composition comprises a VL comprising or consisting of SEQ ID NO:13. In certain cases, the anti-human tau antibody of the pharmaceutical composition comprises a VH comprising or consisting of SEQ ID NO:12 and a VL comprising or consisting of SEQ ID NO:13.

In certain cases, the anti-human tau antibody of the pharmaceutical composition comprises a heavy chain comprising or consisting of SEQ ID NO:14. In certain cases, the anti-human tau antibody of the pharmaceutical composition comprises a light chain comprising or consisting of SEQ ID NO:15. In certain cases, the anti-human tau antibody of the pharmaceutical composition comprises a heavy chain comprising or consisting of SEQ ID NO:14 and a light chain comprising or consisting of SEQ ID NO:15.

### Indications

The anti-human tau antibodies described herein are expected to be useful in the treatment of tauopathies. Tauopathies are a class of neurodegenerative diseases associated with the pathological aggregation of tau protein in neurofibrillary or gliofibrillary tangles in the human brain.

Exemplary tauopathies include progressive supranuclear palsy, Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, globular glial tauopathy, subacute sclerosing panencephalitis, Tangle only dementia, multi-infarct dementia, stroke, chronic traumatic encephalopathy, traumatic brain injury, concussion, seizures, epilepsy, and acute lead encephalopathy.

In one embodiment, the anti-human tau antibodies described herein are used to treat progressive supranuclear palsy.

In another embodiment, the anti-human tau antibodies described herein are used to treat Alzheimer's disease.

### Methods of Treatment

The disclosure features methods of treating human subjects with a tauopathy (see above) with an anti-human tau antibody disclosed herein or a pharmaceutical composition disclosed herein. In one embodiment, the tauopathy is progressive supranuclear palsy. In another embodiment, the tauopathy is Alzheimer's disease.

In certain embodiments, the method comprises administering to the human subject in need thereof an anti-human tau antibody in an amount effective to reduce significantly the level of tau (e.g., total Tau and/or free Tau) in an extracellular fluid (e.g., cerebrospinal fluid (CSF), interstitial fluid (ISF), blood, or a blood fraction (e.g., serum or plasma)) in the individual. “Free Tau” refers to a tau polypeptide that is not bound to an anti-human tau antibody. In one embodiment, the free Tau is extracellular Tau (eTau). “Total Tau” includes free Tau and Tau that is bound to an anti-human tau antibody. In one particular embodiment, the method comprises administering to the human subject in need thereof an anti-human tau antibody in an amount effective to reduce significantly the level of free eTau. In some embodiments, the level of tau (e.g., total Tau and/or free Tau) is significantly reduced within 36 hours of administration of the anti-human tau antibody. In some embodiments, the level of tau (e.g., total Tau and/or free Tau) is significantly reduced within 24 hours of administration of the anti-human tau antibody. In some embodiments, the level of free eTau is significantly reduced within 24 hours of administration of the anti-human tau antibody. In some cases, an effective amount of an anti-human tau antibody is an amount that is effective to reduce significantly the level of tau (e.g., total Tau and/or free Tau) in an extracellular fluid within 48 hours, 36 hours, within 24 hours, within 12 hours, within 8 hours, within 4 hours, within 2 hours, within 1 hour, within 30 minutes, within 15 minutes, or within 5 minutes, of administration of the anti-human tau antibody. For example, in some cases, an effective amount of an anti-human tau antibody is an amount that is effective to reduce significantly the level of Tau (e.g., total Tau and/or free Tau) in an extracellular fluid within from 5 minutes to about 10 minutes, from about 10 minutes to about 15 minutes, from about 15 minutes to about 30 minutes, from about 30 minutes to about 1 hour, from about 1 hour to about 2 hours, from about 2 hours to about 4 hours, from about 4 hours to about 8 hours, from about 8 hours to about 12 hours, from about 12 hours to about 24 hours, from about 24 hours to about 36 hours, from about 24 to about 48 hours, or from about 36 hours to about 48 hours.

A significant reduction in the level of tau (e.g., total Tau and/or free Tau) in an extracellular fluid (e.g., CSF, ISF, blood, or a blood fraction (e.g., serum or plasma)) of an individual is an at least 30% reduction, at least 35% reduction, at least 40% reduction, at least 45% reduction, at least 50% reduction, an at least 55% reduction, an at least 60% reduction, an at least 65% reduction, an at least 70% reduction, an at least 75% reduction, an at least 80% reduction, an at least 85% reduction, an at least 90% reduction, an at least 95% reduction, or a greater than 90% reduction. In some instances, the significant reduction is a statistically significant reduction. In some instances, the significant reduction is a clinically significant reduction. In some embodiments, the level of tau (e.g., total Tau and/or free Tau) in an extracellular fluid is reduced to a normal, control level (e.g., about 100 pg/ml). In some embodiments, the level of Tau (e.g., total Tau and/or free Tau) in an extracellular fluid is reduced to an undetectable level. In some cases, the extracellular fluid is CSF. In other cases, the extracellular fluid is ISF. In other cases, the extracellular fluid is plasma. In other cases, the extracellular fluid is whole blood. In other cases, the extracellular fluid is serum.

In certain instances, an anti-human tau antibody described herein is administered to the human subject at a fixed dose of 2000 mg. In certain instances, an anti-human tau antibody is administered to the human subject at a fixed dose of 2100 mg. In other instances, an anti-human tau antibody is administered to the human subject at a fixed dose of 150 mg. In further instances, an anti-human tau antibody is administered to the human subject at a fixed dose of 4200 mg. In certain embodiments, the above-noted fixed doses of an anti-human tau antibody described herein are administered to the human subject once every four weeks.

In certain cases, an anti-human tau antibody described herein is administered to the human subject as part of a pharmaceutical composition. In one embodiment, the pharmaceutical composition comprises 50 mg/mL of the anti-human tau antibody, 20 mM histidine, 250 mM sucrose, and 50 μM DTPA. The pharmaceutical composition has a pH of 6.0. In certain embodiments, the pharmaceutical composition is administered to the human subject in an amount sufficient to deliver a fixed dose of 2000 mg of the anti-human tau antibody. In certain embodiments, the pharmaceutical composition is administered to the human subject in an amount sufficient to deliver a fixed dose of 2100 mg of the anti-human tau antibody. In certain embodiments, the pharmaceutical composition is administered to the human subject in an amount sufficient to deliver a fixed dose of 700 mg of the anti-human tau antibody. In certain embodiments, the pharmaceutical composition is administered to the human subject in an amount sufficient to deliver a fixed dose of 150 mg of the anti-human tau antibody. In certain embodiments, the pharmaceutical composition is administered to the human subject in an amount sufficient to deliver a fixed dose of 210 mg of the anti-human tau antibody. In certain embodiments, the pharmaceutical composition is administered to the human subject in an amount sufficient to deliver a fixed dose of 4200 mg of the anti-human tau antibody.

In certain embodiments of these methods, the anti-human tau antibody is administered to the human subject in need thereof by an intravenous route.

In certain embodiments, the anti-human tau antibody comprises an immunoglobulin heavy chain variable region (VH) and an immunoglobulin light chain variable region (VL), wherein the VH comprises VH complementarity determining regions (VH-CDRs), wherein VH-CDR1 comprises or consists of the amino acid sequence of SEQ ID NO:16; VH-CDR2 comprises or consists of the amino acid sequence of SEQ ID NO:17; and VH-CDR3 comprises or consists of the amino acid sequence of SEQ ID NO:18; and the VL comprises VL-CDRs, wherein VL-CDR1 comprises or consists of the amino acid sequence of SEQ ID NO:19; VL-CDR2 comprises or consists of the amino acid sequence of SEQ ID NO:20; and VL-CDR3 comprises or consists of the amino acid sequence of SEQ ID NO:21.

In certain embodiments, the VH of the anti-human tau antibody comprises or consists of SEQ ID NO:12 and the VL comprises or consists of SEQ ID NO:13.

In certain embodiments, the anti-human tau antibody comprises a heavy chain and a light chain, wherein the heavy chain comprises or consists of SEQ ID NO:14 and the light chain comprises or consists of SEQ ID NO:15.

The following example is not to be construed as limiting the scope of the invention in any way.

### EXAMPLES

### Example 1: A Single Ascending Dose Study of an Anti-Human Tau Antibody, BIIB092

BIIB092 is a humanized antibody that recognizes human extracellular Tau (eTau). The purposes of this study was to evaluate the safety, tolerability, and pharmacokinetics (PK) of BIIB092 as well as the pharmacodynamic (PD) effects of BIIB092 on extracellular tau (eTau) after a single intravenous (IV) infusion of BIIB092 in healthy human subjects. Specifically, BIIB092 was tested to determine its efficacy in preventing transmission of tau pathology by binding and reducing free eTau in human CSF.

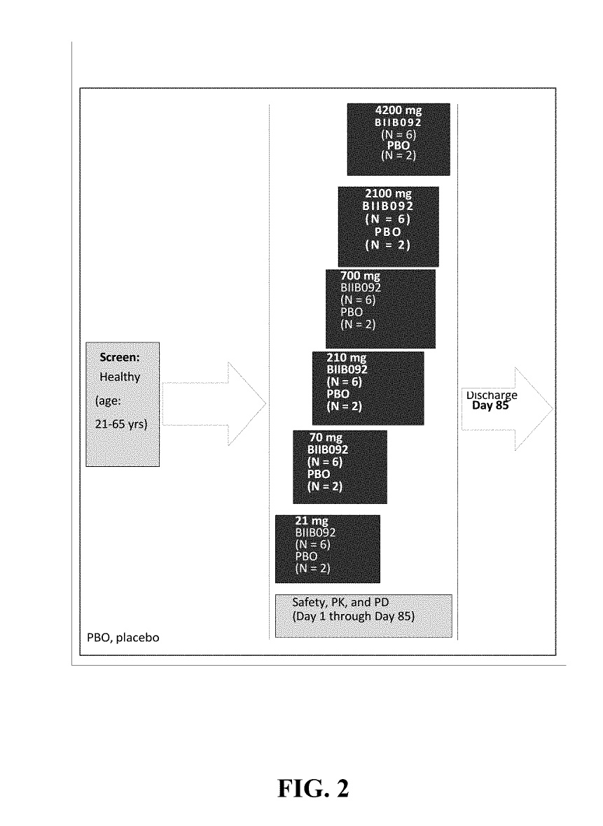

This study was a randomized, double blind, placebo controlled single ascending dose trial. Healthy subjects (age: 21-65) in 6 ascending dose cohorts (21 mg, 70 mg, 210 mg, 700 mg, 2100 mg and 4200 mg of BIIB092) comprised of 8 subjects per cohort were administered a single intravenous (IV) infusion of BIIB092 (6 subjects) or placebo (2 subjects). See, FIG. 2. Safety assessments, and serum and CSF samples (including 4 lumbar punctures) were collected over 12 weeks. Pharmacokinetic parameters (in serum and CSF) and pharmacodynamic measures (CSF concentrations of free eTau) and corresponding change and percent change from baseline were evaluated.

Increases in peak (Cmax) and exposure (AUC[INF]) of BIIB092 in serum appeared to be dose proportional. The terminal elimination half-life of BIIB092 was approximately 25 days. CSF concentrations of BIIB092 increased with dose and appeared dose-proportional. CSF-to-serum ratio of BIIB092 was approximately 0.2% and similar across the dose range. Most adverse events were mild. There were no serious adverse events or discontinuations due to adverse events. The extent and duration of suppression of eTau increased with dose. Following single doses of BIIB092, suppression of CSF eTau at 28 days ranged from 65% to 96% at doses ranging from 70 mg to 4200 mg.

The ability of BIIB092 to robustly suppress CSF concentrations of free eTau in this phase 1 study suggests that BIIB092 has utility for the treatment of human tauopathies (e.g., progressive supranuclear palsy). Single doses of BIIB092 administration were safe and well tolerated at doses up to 4200 mg.

### Example 2: Bayesian Eₘₐₓ Model

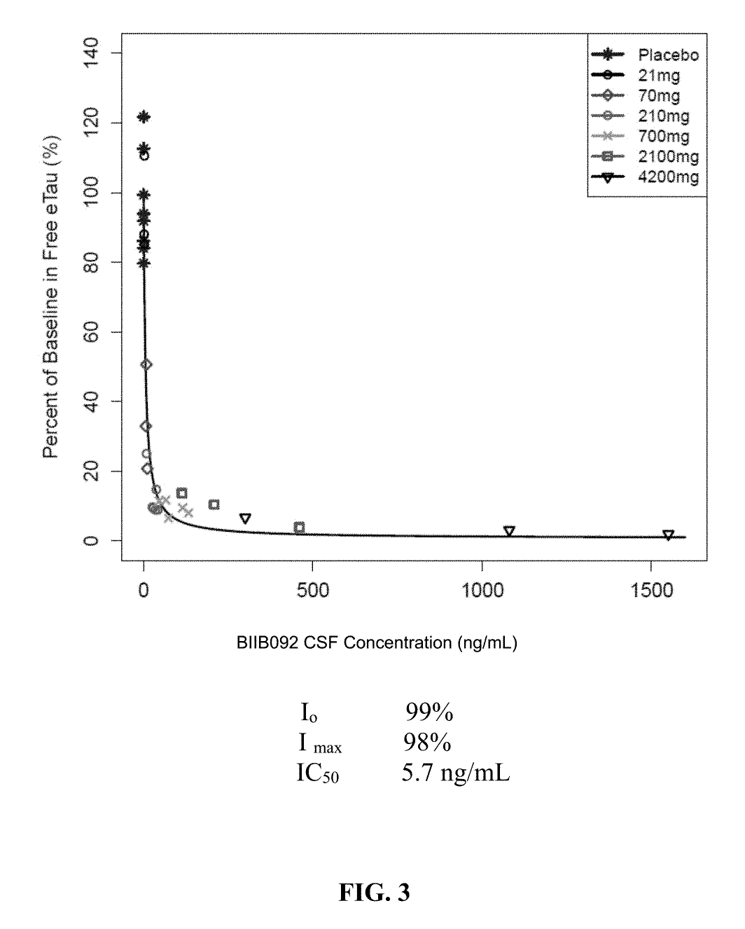

An exposure-response model (Bayesian Eₘₐₓ) of CSF concentration versus eTau suppression was constructed (see, FIG. 3). The Bayesian Eₘₐₓ model captured the observed eTau suppression reasonably well.

### Example 3: Multiple Ascending Dose Study of an Anti-Human Tau Antibody, BIIB092, in Patients with Progressive Supranuclear Palsy

The purpose of this study was to assess the safety, tolerability, pharmacokinetic (PK) and pharmacodynamic (PD) effects of BIIB092 on free extracellular tau (eTau) after intravenous (IV) infusions of BIIB092 every 4 weeks (Q4W) in patients with progressive supranuclear palsy (PSP). Specifically, this study was designed to evaluate the safety profile of BIIB092 and its ability to reduce free eTau in the CSF of patients with PSP.

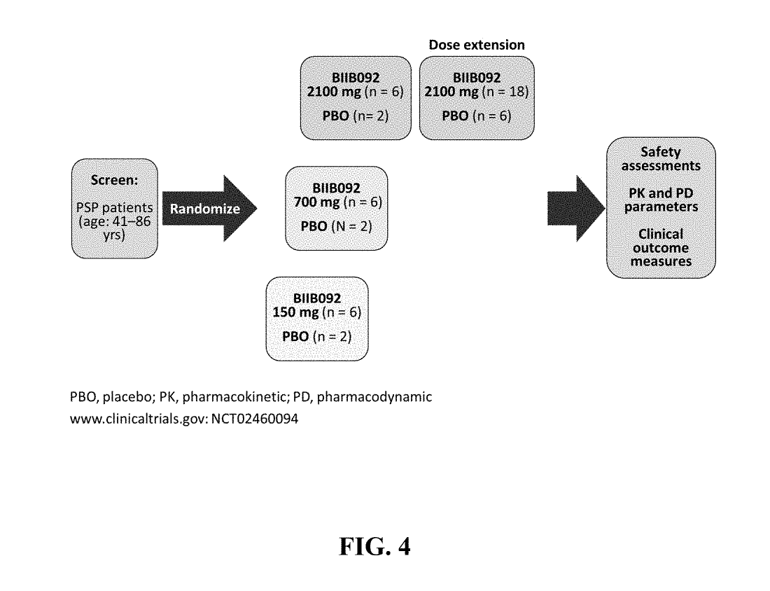

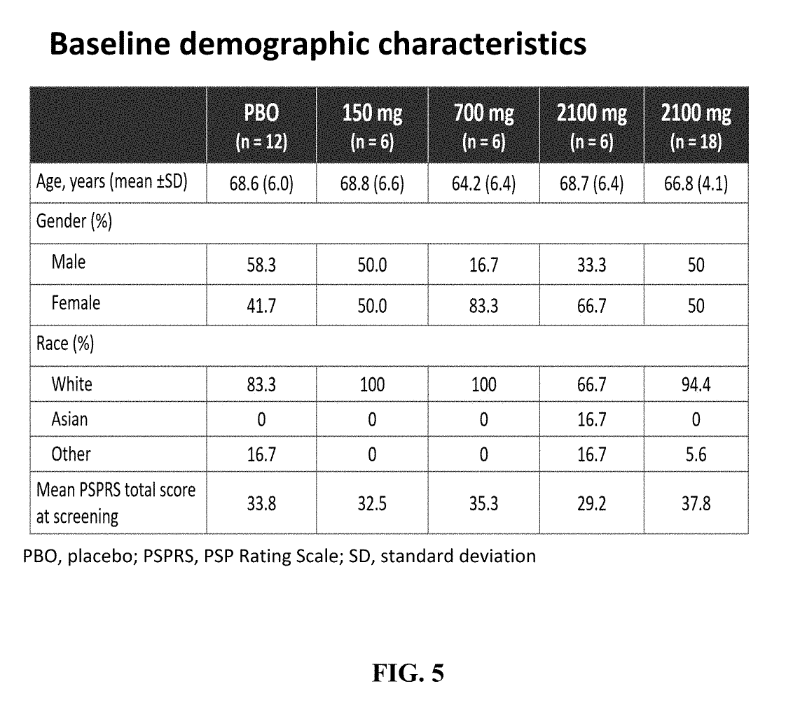

The baseline demographic characteristics for this study are shown in FIG. 4. This study was a randomized, double-blind, placebo-controlled, multiple ascending dose trial of 48 patients with PSP, of whom 12 (25%) received placebo. Three ascending dose panels (150 mg, 700 mg, and 2100 mg) comprised of 8 patients per panel, were administered IV infusions of BIIB092 (6 patients) or placebo (2 patients) Q4W for 12 weeks; an additional 24 patients were treated with BIIB092 at a dose of 2100 mg (18 patients) or placebo (6 patients) administered Q4W for 12 weeks. See, FIG. 5. All patients were also offered the opportunity to participate in an open-label extension study. Safety assessments and serum and CSF samples were collected over the 12 weeks. PK parameters (in serum and CSF), PD measures (concentrations of CSF free eTau), and corresponding change and percent change from baseline were evaluated. Clinical outcome measures including the PSP Rating Scale and Schwab and England Activities of Daily Living Scale were also employed.

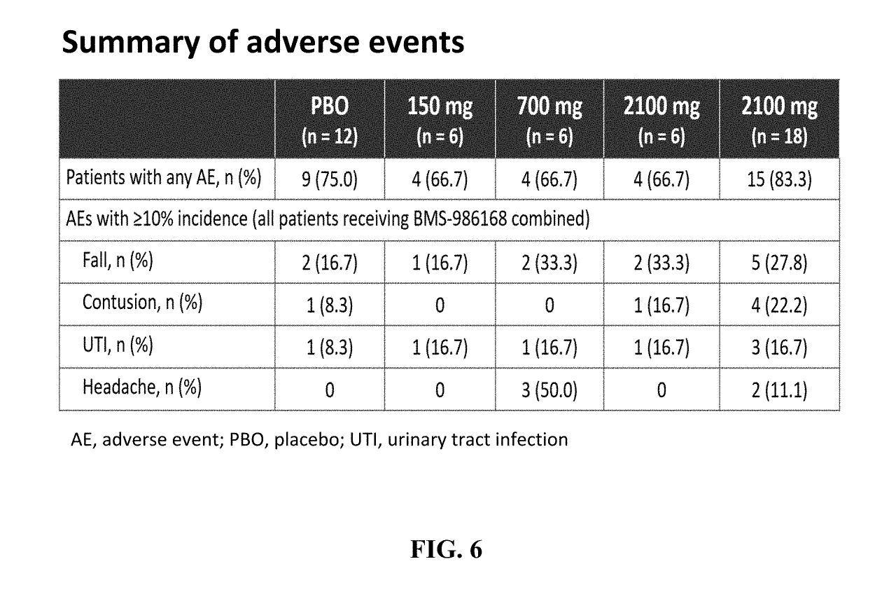

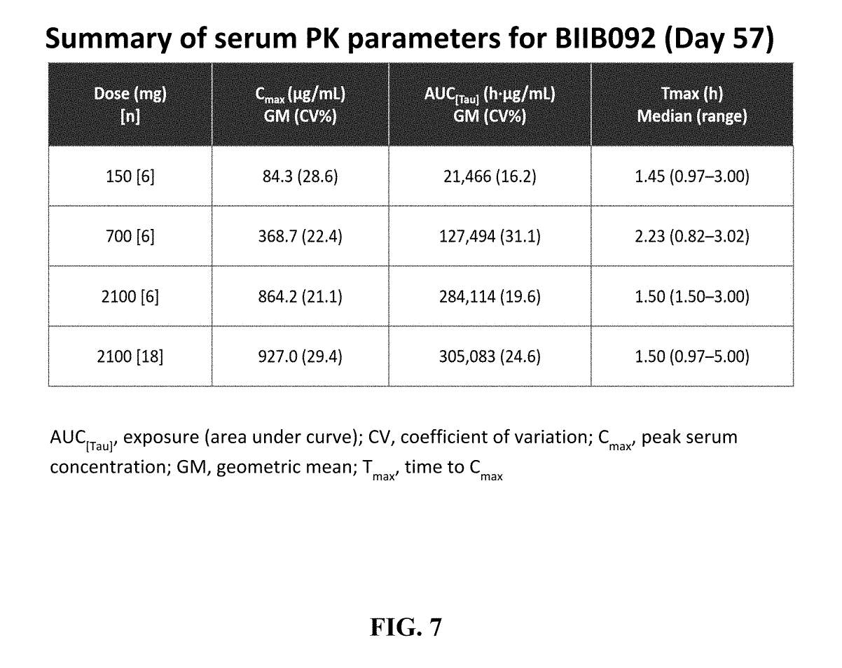

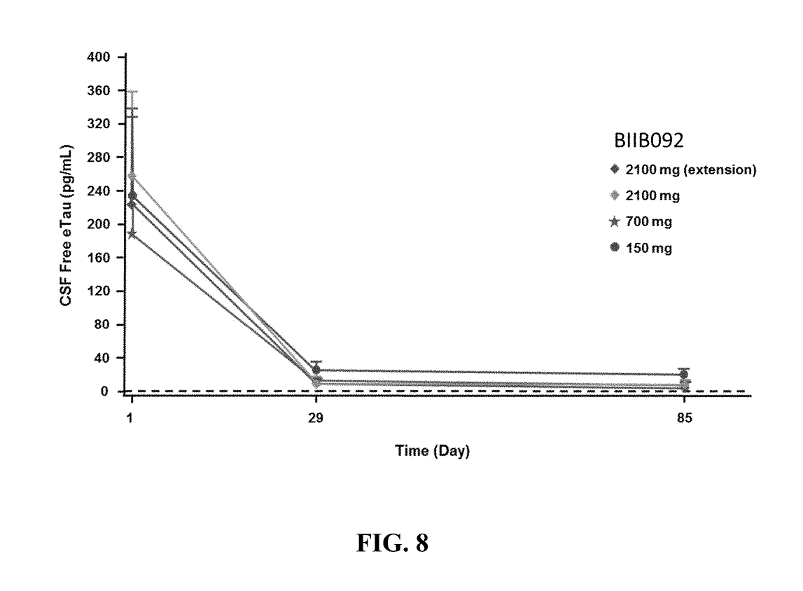

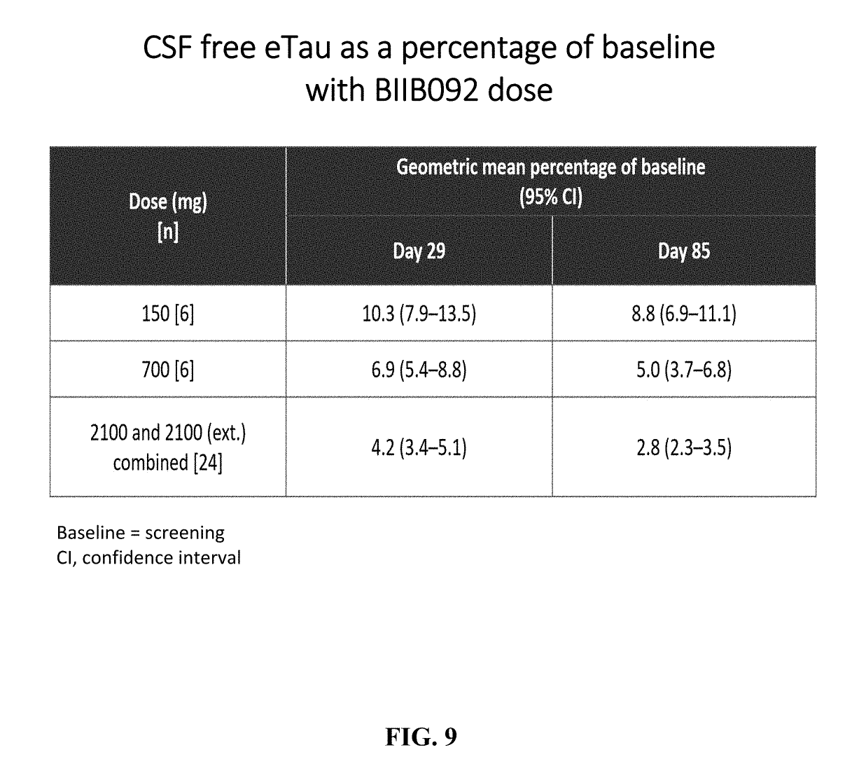

Patients' mean age was 67.4±5.5 years; 54.2% were female. Concentrations of BIIB092 in serum and CSF increased with dose. The percentages of patients experiencing adverse events (AEs) were similar in the BIIB092 and placebo groups (˜75%). Most AEs were mild. See, FIG. 6. There were no deaths or discontinuations due to AEs. A summary of the serum PK parameters for BIIB092 is provided in FIG. 7. Mean suppression of CSF free eTau was approximately 90-96% (Day 29) and 91-97% (Day 85) at doses ranging from 150 mg to 2100 mg. Although CSF and serum exposures and reductions of CSF free eTau increased with BIIB092 dosage, significant reductions of CSF free eTau were observed with all dosages employed in the study. See, FIGS. 8 and 9.

Administration of multiple doses of BIIB092 was safe and well tolerated at doses up to 2100 mg in patients with PSP.

### Example 4: BIIB092 Dose Selection Based on Simulated PK and eTau Suppression

Using the estimated PK-PD model parameters and the variability around these parameters in PSP patients, 1000 profiles were simulated and the time course of PK in serum and CSF and PD (eTau) in CSF were obtained for two doses, namely 2000 mg and 700 mg of BIIB092 administered once every 4 weeks (Q4W). eTau concentrations were converted to percent suppression, relative to each subject's baseline value, prior to summarization based on the simulations. Table 1 below shows summary statistics of percent suppression of eTau relative to baseline levels at 4 weeks, 12 weeks, 24 weeks, and 48 weeks following the 2000 mg Q4W dosing regimen.

TABLE 1
Summary Statistics of Percent Suppression of eTau Relative to
Baseline Following 2000 mg Q4W (Simulated)

| Statistic | 4 Weeks | 12 Weeks | 24 Weeks | 48 Weeks |
|---|---|---|---|---|
| N | 1000 | 1000 | 1000 | 1000 |
| Median | 96.46 | 97.46 | 97.57 | 97.58 |
| Minimum | 93.87 | 94.80 | 94.82 | 94.82 |
| Maximum | 98.25 | 98.92 | 99.03 | 99.04 |
| 10th percentile | 95.35 | 96.34 | 96.43 | 96.43 |
| 90th percentile | 97.37 | 98.25 | 98.39 | 98.40 |

Table 2 shows summary statistics of percent suppression of eTau relative to baseline levels at 4 weeks, 12 weeks, 24 weeks, and 48 weeks following the 700 mg Q4W dosing regimen.

TABLE 2
Summary Statistics of Percent Suppression of eTau Relative to
Baseline Following 700 mg Q4W (Simulated)

| Statistic | 4 Weeks | 12 Weeks | 24 Weeks | 48 Weeks |
|---|---|---|---|---|
| N | 1000 | 1000 | 1000 | 1000 |
| Median | 93.28 | 94.75 | 94.94 | 94.95 |
| Minimum | 88.75 | 89.97 | 90.04 | 90.04 |
| Maximum | 96.31 | 97.68 | 97.96 | 98.00 |
| 10th percentile | 91.85 | 93.15 | 93.27 | 93.28 |
| 90th percentile | 94.58 | 96.02 | 96.28 | 96.30 |

Both 2000 mg and 700 mg doses of BIIB092, administered Q4W lead to robust suppression of eTau at trough. The 2000 mg dose of BIIB092 is associated with slightly higher suppression (3 to 5%) at trough than the 700 mg dose. Ninety percent of all subjects that are dosed with the 2000 mg Q4W dose are expected to have a percentage suppression of eTau at or above 96% at trough. Ninety percent of all subjects that are dosed the 700 mg Q4W dose are expected have a percentage suppression of eTau at or above 93% at trough.

Given the robust and persistent suppression of eTau up to 12 weeks postdose at doses at and above 210 mg that were studied, and the ˜2× higher CSF concentrations of BIIB092 observed in PSP patients compared to healthy subjects, dosing BIIB092 can also be done on a less frequent basis, e.g., once every 12 weeks (Q12W). Simulations were performed using the same PK-PD model for a 2000 mg dose, administered Q12W. eTau suppression is summarized in Table 3.

TABLE 3
Summary Statistics of Percent Suppression of eTau Relative to
Baseline Following 2000 mg Q12W (Simulated)

| | 4 | 12 Weeks | 24 Weeks | | 48 Weeks |
|---|---|---|---|---|---|
| Statistic | Weeks | (trough) | (trough) | 28 Weeks | (trough) |
| N | 1000 | 1000 | 1000 | 1000 | 1000 |
| Median | 96.47 | 90.08 | 90.35 | 96.64 | 90.36 |
| Minimum | 93.46 | 74.05 | 74.13 | 93.54 | 74.13 |
| Maximum | 98.22 | 95.54 | 96.05 | 98.37 | 96.15 |
| 10th percentile | 95.35 | 86.01 | 86.20 | 95.46 | 86.21 |
| 90th percentile | 97.37 | 92.79 | 93.11 | 97.54 | 93.15 |

Ninety percent of all subjects that are administered the 2000 mg Q12W dose are expected have a percentage suppression of eTau at or above 86% at trough (i.e., end of the 12-week dosing interval). However, subjects are expected to be at or above 95% suppression during the one month immediate to the infusion, with slightly attenuated suppression in the ensuing 2 months. Overall, Q12W dosing is also expected to be associated with robust and persistent lowering of eTau and may be preferred by patients and caregivers.

### Example 5: BIIB092 Formulation Optimization for Excipient Content

In this excipient optimization stability, BIIB092 was evaluated at 10 mg/mL in 20 mM histidine buffer at pH 5.5, 6.0, and 6.5, containing 0.05% PS-80 and 50 μM DTPA, plus either 250 mM sucrose or 250 mM sorbitol. In addition, BIIB092 was evaluated at 20 mg/mL and 50 mg/mL in 20 mM histidine at pH 6.0 containing 0.05% PS-80, 50 μM DTPA, and 250 mM sucrose. A list of the formulations is shown in Table 4.

TABLE 4
Summary of Formulations for BIIB092
Excipient Optimization Study

| | | | Su- | Sor- | | | |
|---|---|---|---|---|---|---|---|
| Formulation | | | crose | bitol | PS-80 | DTPA | API Conc. |
| Abbreviation | Buffer | pH | (mM) | (mM) | (%) | (μM) | (mg/mL) |
| E1 | 20 mM | 5.5 | 250 | 0 | 0.05 | 50 | 10 |
| E2 | 20 mM | 6.0 | 250 | 0 | 0.05 | 50 | 10 |
| E3 | 20 mM | 6.5 | 250 | 0 | 0.05 | 50 | 10 |
| E4 | 20 mM | 5.5 | 0 | 250 | 0.05 | 50 | 10 |
| E5 | 20 mM | 6.0 | 0 | 250 | 0.05 | 50 | 10 |
| E6 | 20 mM | 6.5 | 0 | 250 | 0.05 | 50 | 10 |
| E7 | 20 mM | 6.0 | 250 | 0 | 0.05 | 50 | 20 |
| E8 | 20 mM | 6.0 | 250 | 0 | 0.05 | 50 | 50 |

All formulations were stored at 40±2° C./75±5% RH for up to three months and at 5±3° C. and 25±2° C./60±5% RH up to 12 months. Initial and time point samples were evaluated for API stability by appearance, pH, A₂₈₀, HIAC, SEC, CEX, and potency ELISA. Peptide mapping analysis was performed only at the initial, two month, and 12-month time points. Microflow imaging was evaluated only at the three-month time point. The test methods are shown in Table 5 and summary of testing at each time point is shown in Table 6.

TABLE 5
Analytical Testing for BIIB092 Excipient Optimization Study

| Method | Result Reporting |
|---|---|
| Appearance | physical state, color, clarity |
| pH | pH |
| Protein Content | mg/mL |
| by UV | |
| CEX | % acidic variants, % main peak, % basic variants |
| SEC | % monomer, % HMW, % LMW |
| HIAC | cumulative counts per mL at ≥2 μm, ≥5 μm, ≥10 μm, |
| | ≥25 μm |
| MFI | Particulates (≥2 μm, ≥5 μm, ≥5 μm with AR ≥ 0.85, |
| | ≥10 μm, ≥25 μm) |
| Potency ELISA | % Relative Potency³ |
| Peptide Mapping | IPN007 chemical degradation³ |

¹Testing was performed on 3 × 0.5 mL runs, included data from all three runs
²MFI was only tested and reported at the three-month time point
³ELISA and peptide mapping testing were performed only at the initial, two month, and final time points

TABLE 6
Pull Schedule for BIIB092 Excipient Optimization Study

| | | Months | | | | | |
|---|---|---|---|---|---|---|---|
| Storage Condition | Initial | 1 | 2 | 3 | 6 | 9 | 12 |
| 5 ± 3° C. | A | B | A | C | D | D | E |
| 25 ± 2° C./60 ± 5% RH | | B | A | C | D | D | E |
| 40 ± 2° C./75 ± 5% RH | | B | A | C | | | |

A = Testing according to Table 2, excluding MFI.
B = Testing according to Table 2, excluding ELISA, MFI and peptide map.
C = Testing according to Table 2, excluding ELISA and peptide map.
D = Testing of formulations E1-E3, E7, and E8 according to Table 2, except MFI, ELISA and peptide map.
E = Testing of formulations E1-E3, E7, and E8 according to Table 2, except MFI.

### Appearance

All samples at all time points and conditions except for one were assessed to be clear, colorless solutions, free of any visible product-related particulates. One sample (E3, six month, 25±2° C./60° 5% RH) was noted to contain a single large white particle, which was considered to be a contaminant and prevented further testing of that particular sample.

### Measurement of pH

All samples at all time points and conditions exhibited pH values that were within ±0.1 pH units of the nominal values for each formulation. Any observed differences in pH were therefore within the variability of the method.

### Protein Content by Ultraviolet/Visible Spectroscopy

All samples at all time points and conditions exhibited protein concentrations that did not differ significantly from their respective initial values. Formulations targeted to 10 mg/mL (E1-E6) all exhibited protein concentrations between 9.8-11.4 mg/mL. Note that the measured concentrations of the sorbitol formulations (E4-E6) were approximately 0.5 mg/mL higher than those of the sucrose formulations (E1-E3), but this merely reflects slightly higher concentrations from sample preparation. Formulation E7 (targeted to 20 mg/mL) ranged from 19.0-20.2 mg/mL, and E8 (targeted to 50 mg/mL) ranged from 50.0-53.7 mg/mL. Protein content by A280 demonstrated no significant trends in protein concentration throughout the stability time points for any of the formulations tested.

### Particle Count (HIAC)

In all formulations, increases in the number of larger particles (10 μm and 25 μm) were observed across the stability time points. However, there was no dependence of particle formation on storage temperature. Formulations E1-E3 exhibited overall increases in large particle counts out to the 12-month time point; any differences between the three formulations are likely due to the variability of the method. While the trends were not extremely strong, relative to the noise, it should be noted that the absolute magnitude of counts was considerable in the samples. Particle increases were especially pronounced in the E7 and E8 formulations (with 20 mg/mL and 50 mg/mL of API, respectively); particles at 10 μm in E7 increased from 12 counts at the initial time point to 1161 counts for the nine month/25° C./60% RH sample, particles at 10 μm in E8 increased from 18 counts at the initial time point to 1003 counts for the nine month/25° C./60% RH sample. Particle counts in these two samples at other longer range stability time points and conditions were also much higher than the initial particle counts. Differences between storage temperatures are likely due to variability of the method. For the formulations E4-E6, which were only tested out to three months, particle counts out to that time point were comparable to those observed in E1-E3, and smaller than those observed in E7-E8.

### Size Exclusion Chromatography

At each time point, small but significant decreases in percent HMW were observed for all formulations as stability condition temperature increased. Generally, the temperature-dependent differences in HMW impurities were smaller for formulations (E1-E3) prepared with 250 mM sucrose than for formulations (E4-E6) prepared with 250 mM sorbitol. At the 12-month time point, formulations E2 and E3 exhibited the highest main peak purity, indicating the least aggregation and fragmentation of the API in these formulations.

Within the time point data for each formulation, there is no solid evidence for increasing HMW content at any storage condition. The difficulty in comparing time points makes a more distinct conclusion difficult. However, some comparison can be made between time points, when the reference standard performance in each sample queue is comparable. Two such time points were the initial and final (12 month) time points, for which all reference standard injections exhibited 1.5±0.1% HMW. At these two time points, there was little difference in HMW content for the study samples.

For each time point, the percent LMW did not appear to change significantly (increases of between 0.1-0.3% for some formulations and time points/conditions, others saw no change in percent LMW).

### Cation Exchange Chromatography

Percent acidic variants increased over time for all formulations, and demonstrated greater increases with increasing temperature, and appeared to be lower with lower pH. Conversely, percent basic variants, while increasing over time and with higher stability temperature, demonstrated lower variants with increasing pH. There did not appear to be any significant differences in either percent acidic or percent basic variants in samples formulated at higher concentrations (E7-20 mg/mL, E8-50 mg/mL) versus comparable sample at 10 mg/mL at the same pH (6.0).

Overall, the percent main peak for the samples that were tested out to twelve months (E1-E3, E7-E8) appeared to be comparable for those samples at all concentrations that were formulated at pH 5.5 and 6.0. The sample formulated at pH 6.5 (E3) exhibited somewhat lower percent main peak versus those samples at lower pH (70.6% for E3 versus 76.9% for E2 at 12 month/25° C./60% RH).

### Potency by Enzyme-Linked Immunosorbent Assay

Samples demonstrated potency determinations ranging from 82-126%. There did not appear to be any trends in percent potency with respect to either formulation type or stability time points/conditions.

### Peptide Mapping

Peptide maps were interpreted in light of the identifications made by LC/MS. For deamidation of asparagine at light chain site N33 or N35, no significant differences from frozen reference standard were observed in formulations stored at 5° C., even out to 12 months. At room temperature (25° C./60% RH), deamidation above background could be detected in the pH 6.5 formulations (E3 and E6) at two months. Significant deamidation (>2% increase versus reference) was observed in a pH-dependent manner after 12 months at 25° C./60% RH. Similar increases were observed after two months at 40° C./75% RH, with a clear dependence on pH, but with equivalent results in sorbitol and sucrose. Note that levels of modification in reference standard measured by UV (6.1-8.3%) were reasonably similar to those measured for the parent material by LC/MS (3.0%).

Deamidation of asparagine at heavy chain site N381 or N386 responded very similarly to the light chain deamidation site. No significant differences from frozen reference standard were observed in formulations stored at 5° C., even out to 12 months. At room temperature (25° C./60% RH), deamidation above background could be detected in the pH 6.5 formulations (E3 and E6) at two months. Significant deamidation (>2% increase versus reference) was observed in a pH-dependent manner after 12 months at 25° C./60% RH. Similar increases were observed after two months at 40° C./75% RH, with a clear dependence on pH, but with equivalent results in sorbitol and sucrose. The levels of modification in reference standard measured by UV (5.9-6.8%) were similar to those measured for the parent material by LC/MS (3.3%).

For deamidation of asparagine at heavy chain site N312, no significant differences from frozen reference standard were observed in the study overall. Even at the 25° C./60% RH, 12 month and 40° C./75% RH, two-month time points, the peak percentages were not significantly different from the frozen reference standard. This may indicate that this asparagine site is not susceptible to deamidation, or that additional species are present at the migration times of the peak identified as modified asparagine, masking the deamidation response. In support of this theory, total N312 modification was somewhat higher in reference standard by UV analysis (5.1-9.8%) than in the parent material by LC/MS (2.8%). Because the succinimide form of the Asn/Asp intermediate was most abundant in the LC/MS data, actual deamidation may simply be low (<1%) at all time points.

Proline and lysine hydroxylation, although present in significant abundance in the reformulated samples, did not appear to change with time, regardless of storage temperature. Hydroxylation of P189 was very consistent and unchanged at all time points. Interestingly, this proline hydroxylation was lower in the frozen reference standard. The levels of proline hydroxylation in reference standard measured by UV (1.1-1.6%) were reasonably similar to those measured by LC/MS (1.0%). Measurement of K121 hydroxylation produced much larger differences in quantities. However, because no consistent trends could be observed, it is concluded these differences represent analytical noise. The levels of lysine hydroxylation in reference standard measured by UV (4.1-7.9%) were reasonably similar to those measured in the parent material by LC/MS (2.8%).

Methionine oxidation at heavy chain site M425 was fairly conducive to analysis. Small, but consistent, levels of oxidation were seen in all formulations. Oxidation increased slowly as a function of time and temperature, but was not affected by the pH or formulation components. Even after two months at 40° C./75% RH, the oxidation increased only by about 0.5%. The levels of modification in reference standard measured by UV (0.2-0.6%) were very similar to those measured in the parent material by LC/MS (0.3%).

### Particle Count (Micro-Flow Imaging)

Particle counting by WI was performed only at the three-month time point. Overall, samples formulated with 250 mM sorbitol appeared to demonstrate lower particle counts at larger particle sizes (10 μm and 25 μm). Among those samples formulated in 250 mM sucrose, formulation E2 appeared to demonstrate slightly lower particle counts overall. There did not appear to be a significant difference in particle counts between formulations at differing API concentration.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a tauopathy in a human subject in need thereof, the method comprising intravenously administering to the human subject a fixed dose of 2000 mg of an anti-human tau antibody once every four weeks, wherein the anti-human tau antibody comprises an immunoglobulin heavy chain variable region (VH) and an immunoglobulin light chain variable region (VL), wherein:
(a) the VH comprises VH complementarity determining regions (VH-CDRs), wherein:
VH-CDR1 consists of the amino acid sequence of SEQ ID NO:16;
VH-CDR2 consists of the amino acid sequence of SEQ ID NO:17; and
VH-CDR3 consists of the amino acid sequence of SEQ ID NO:18; and
(b) the VL comprises VL-CDRs, wherein:
VL-CDR1 consists of the amino acid sequence of SEQ ID NO:19;
VL-CDR2 consists of the amino acid sequence of SEQ ID NO:20; and
VL-CDR3 consists of the amino acid sequence of SEQ ID NO:21.

2. The method of claim 1, wherein the tauopathy is Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia, stroke, chronic traumatic encephalopathy, traumatic brain injury, concussion, seizures, epilepsy, or acute lead encephalopathy.

3. The method of claim 1, wherein the tauopathy is progressive supranuclear palsy.

4. The method of claim 1, wherein the tauopathy is Alzheimer's disease.

5. The method of claim 1, wherein the VH consists of SEQ ID NO:12 and the VL consists of SEQ ID NO:13.

6. The method of claim 1, wherein the anti-human tau antibody comprises a heavy chain and a light chain, wherein the heavy chain consists of SEQ ID NO:14 and the light chain consists of SEQ ID NO:15.

7. A pharmaceutical composition comprising:
(i) an anti-human tau antibody at a concentration of 50 mg/ml,
(ii) histidine at a concentration of 20 mM,
(iii) sucrose at a concentration of 250 mM,
(iv) polysorbate-80 at a concentration of 0.05% (w/v), and
(v) 50 μM diethylenetriamine pentaacetic acid (DTPA)
wherein the anti-human tau antibody comprises an immunoglobulin heavy chain variable region (VH) and an immunoglobulin light chain variable region (VL), wherein:
(a) the VH comprises VH complementarity determining regions (VH-CDRs), wherein:
VH-CDR1 consists of the amino acid sequence of SEQ ID NO:16;
VH-CDR2 consists of the amino acid sequence of SEQ ID NO:17; and
VH-CDR3 consists of the amino acid sequence of SEQ ID NO:18; and
(b) the VL comprises VL-CDRs, wherein:
VL-CDR1 consists of the amino acid sequence of SEQ ID NO:19;
VL-CDR2 consists of the amino acid sequence of SEQ ID NO:20; and
VL-CDR3 consists of the amino acid sequence of SEQ ID NO:21, and wherein the composition has a pH of 6.0.

8. The pharmaceutical composition of claim 7, wherein the VH consists of SEQ ID NO:12 and the VL consists of SEQ ID NO:13.

9. The pharmaceutical composition of claim 7, wherein the anti-human tau antibody comprises a heavy chain and a light chain, wherein the heavy chain consists of SEQ ID NO:14 and the light chain consists of SEQ ID NO:15.

10. A method of treating a tauopathy in a human subject in need thereof, the method comprising intravenously administering to the human subject the pharmaceutical composition of claim 7.

11. The method of claim 10, wherein the anti-human tau antibody is administered at a fixed dose of 150 mg once every four weeks.

12. The method of claim 10, wherein the anti-human tau antibody is administered at a fixed dose of 210 mg once every four weeks.

13. The method of claim 10, wherein the anti-human tau antibody is administered at a fixed dose of 700 mg once every four weeks.

14. The method of claim 10, wherein the anti-human tau antibody is administered at a fixed dose of 2000 mg once every four weeks.

15. The method of claim 10, wherein the anti-human tau antibody is administered at a fixed dose of 2100 mg once every four weeks.

16. The method of claim 10, wherein the anti-human tau antibody is administered at a fixed dose of 4200 mg once every four weeks.

17. The method of claim 11, wherein the pharmaceutical composition is administered for at least 12 weeks.

18. The method of claim 10, wherein the tauopathy is Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia, stroke, chronic traumatic encephalopathy, traumatic brain injury, concussion, seizures, epilepsy, or acute lead encephalopathy.

19. The method of claim 10, wherein the tauopathy is progressive supranuclear palsy.

20. The method of claim 10, wherein the tauopathy is Alzheimer's disease.
